(19) United States
(12) Patent Application Publication
(10) Pub. No.: US 2025/0283861 A1
(43) Pub. Date: Sep. 11, 2025

(54) COVER FOR GAS DETECTOR

(71) Applicant: TOYOTA JIDOSHA KABUSHIKI KAISHA, Toyota-shi (JP)

(72) Inventor: Masahiko MORINAGA, Toyota-shi (JP)

(73) Assignee: TOYOTA JIDOSHA KABUSHIKI KAISHA, Toyota-shi (JP)

(21) Appl. No.: 18/982,308

(22) Filed: Dec. 16, 2024

(30) Foreign Application Priority Data
Mar. 5, 2024 (JP) 2024-032698

(51) Int. Cl. G01N 33/00

(52) U.S. Cl. CPC G01N 33/0009

(57) ABSTRACT

A cover for a gas detector mounted on a ceiling surface includes a flat plate portion disposed along the ceiling surface and to which the gas detector is fixed, and a collar portion extending so as to surround the gas detector in the flat plate portion and inclined downward as the distance from the gas detector increases. At least one of the flat plate portion and the collar portion is provided with a plurality of through holes, and the plurality of through holes are arranged so as to surround the gas detector.

### CROSS-REFERENCE TO RELATED APPLICATION

This application claims priority to Japanese Patent Application No. 2024-032698 filed on Mar. 5, 2024, incorporated herein by reference in its entirety.

### BACKGROUND

### 1. Technical Field

The technology disclosed herein relates to a cover for a gas detector.

### 2. Description of Related Art

Japanese Unexamined Patent Application Publication No. 2022-73236 (JP 2022-73236 A) discloses a fuel cell electric vehicle in which a housing space that houses a fuel cell is provided below a cabin of the vehicle. According to JP 2022-73236 A, the housing space is covered with a ceiling surface formed by the bottom of the cabin, and the ceiling surface is provided with a hydrogen sensor that detects a hydrogen concentration of the housing space.

### SUMMARY

In a fuel cell electric vehicle or other equipment including a fuel cell, a portion where hydrogen may leak is not limited to the fuel cell. Hydrogen may leak from the fitting of a hydrogen tank, joints of various pipes, etc. Providing hydrogen detectors corresponding to the portions where hydrogen may leak causes an increase in cost. Therefore, there is a need for improvement to accurately detect hydrogen leakage by using as few hydrogen detectors as possible. Such a problem is not limited to the case of detecting hydrogen, but is also conceivable in various situations where gas is to be detected.

The present specification discloses a cover for a gas detector attached to a ceiling surface.

The cover includes:
a flat plate portion that is disposed along the ceiling surface and to which the gas detector is fixed; and
a collar portion that extends to surround the gas detector on the flat plate portion and is inclined downward away from the gas detector.

In the above configuration, the cover includes the collar portion. Accordingly, when gas leakage occurs in a space including the ceiling surface, the gas drifting around the ceiling surface is easily retained in the vicinity of the gas detector by the action of the collar portion. As a result, the reliability of detection of the leaked gas by one gas detector can be improved.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a view schematically showing a state in which a hydrogen detector is attached to a ceiling of a space;

FIG. 2 shows a cover according to a first embodiment from a lower perspective;

FIG. 3 is a cross-sectional view taken along III-III line of FIG. 2;

FIG. 4 shows a cover according to a second embodiment from a lower perspective;

FIG. 5 shows a cover according to a third embodiment from a lower perspective;

FIG. 6 is a cross-sectional view taken along VI-VI line of FIG. 5; and

FIG. 7 is a graph showing the change in hydrogen concentration with time.

### DETAILED DESCRIPTION OF EMBODIMENTS

Embodiments will be described with reference to the drawings. Each of the drawings is merely an example, and the present embodiment is not limited to the illustrated contents. In addition, since each of the drawings is an example, the illustrated shape is not accurate or a part thereof is omitted.

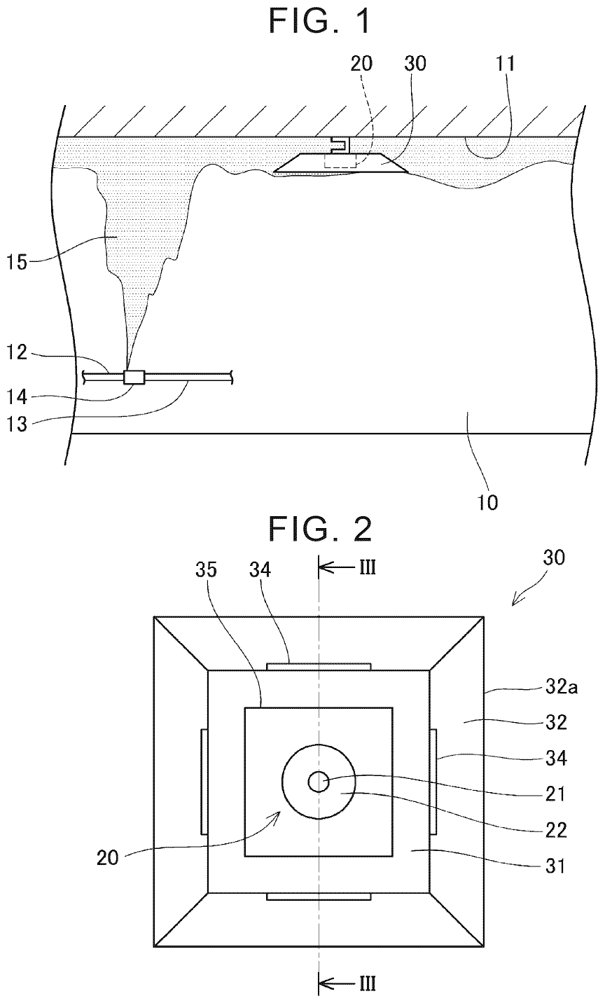

FIG. 1 schematically shows a state in which a hydrogen detector 20 capable of detecting hydrogen is attached to a ceiling surface 11 of a space 10. The hydrogen detector 20 is an example of a “gas detector”. Hereinafter, the hydrogen detector 20 will be described as an example of the gas detector. However, a detector or a sensor that can detect a gas or a gas that is lighter than air and can be detected from the viewpoint of ensuring safety or the like corresponds to the gas detector assumed in the present embodiment.

The space 10 is a space in which a gas to be detected can leak. The ceiling surface 11 is not necessarily a horizontal surface, but is in any case a surface defining an area above the space 10. The space 10 may be a closed space or a partially open space. As an example, the space 10 is a space secured under the floor of a passenger cabin of a fuel cell electric vehicle, and a fuel-cell, a hydrogen-tank, various pipes, and the like are disposed in the space 10.

In FIG. 1, pipes 12 and 13 used for supplying the hydrogen gas 15 and a joint 14 connecting the pipe 12 and the pipe 13 are shown. The joint 14 is an example of a portion where the hydrogen gas 15 may leak. Although not shown in the drawings, it may be understood that the space 10 includes a plurality of locations that can be the source of such leakage. The space 10 is not limited to a space provided in a moving means such as a vehicle, and may be a space secured in various facilities such as a factory, an office, and a home, or a building.

According to FIG. 1, the hydrogen detector 20 is fixed to the cover 30, and the cover 30 is attached to the ceiling surface 11. That is, when the hydrogen detector 20 is said to be attached to the ceiling surface 11, it includes both a case in which at least a part of the hydrogen detector 20 is directly attached to the ceiling surface 11 and a case in which it is indirectly attached to the ceiling surface 11 via the cover 30 or the like. The cover 30 may be referred to as a gas detector cover or the like. As a material for forming the cover 30, various materials such as a metal and a resin are assumed. In consideration of an environment in which the hydrogen detector 20 is installed, durability required for the cover 30, and the like, a suitable material is appropriately selected and the cover 30 is formed. Although not shown in the drawings, the hydrogen detector 20 is connected to cables buried in the ceiling or routed in the space 10 as necessary for communication and power supply securing.

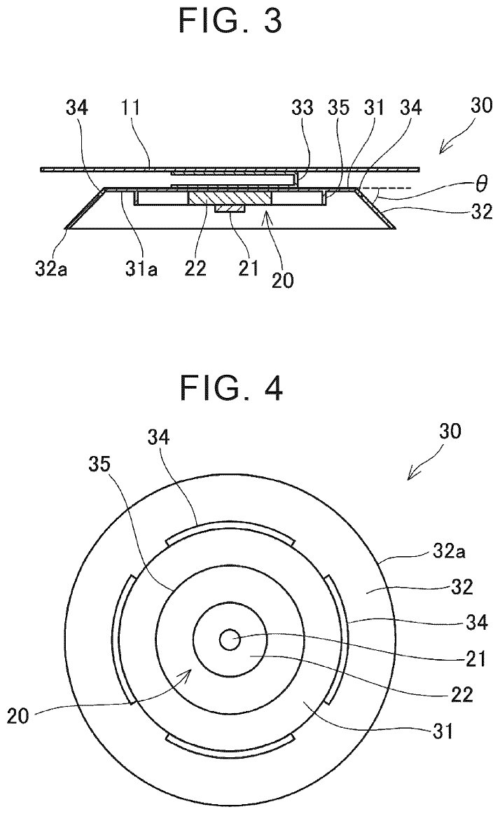

The cover 30 according to the first embodiment will be described with reference to FIGS. 2 and 3.

FIG. 2 shows the cover 30 from a lower perspective. FIG. 3 shows a cross-sectional view through III-III of FIG. 2. According to the first embodiment, the cover 30 generally includes a flat plate portion 31 and a collar portion 32. The flat plate portion 31 is disposed along the ceiling surface 11. Further, the hydrogen detector 20 is fixed to the flat plate portion 31. The term “along the ceiling surface 11” does not refer to only a state parallel to the ceiling surface 11. As described above, the ceiling surface 11 is not necessarily a horizontal surface, and may be a curved surface. Therefore, the flat plate portion 31 only needs to be substantially along the ceiling surface 11, and may be inclined within a predetermined angle range with respect to the ceiling surface 11, for example.

The flat plate portion 31 may be directly fixed to the ceiling surface 11. However, according to the first embodiment, the flat plate portion 31 is fixed to the ceiling surface 11 via the support member 33 in consideration of workability such as screwing at the time of attachment. That is, the support member 33 is fixed to the ceiling surface 11, and the flat plate portion 31 is fixed to the support member 33. The support member 33 is also referred to as a bracket or the like. When the support member 33 is interposed between the ceiling surface 11 and the flat plate portion 31, the flat plate portion 31 is fixed at a position separated from the ceiling surface 11 by a predetermined distance, for example, several centimeters downward. The shape of the support member 33 is not particularly limited. In addition, in the present embodiment, there is no particular limitation on a method of fixing the object and the object.

According to the first embodiment, the flat plate portion 31 has a square shape or a rectangular shape close to a square shape. The flat plate portion 31 may be a flat plate as a whole or may be a member including at least a flat plate-shaped portion. The hydrogen detector 20 includes a sensor unit 21 for detecting hydrogen, and a main body unit 22 that houses a circuit necessary for realizing the function, communication function, and the like of the sensor unit 21. The hydrogen detector 20 is fixed to the lower surface 31a of the flat plate portion 31 with the sensor unit 21 facing downward. In the flat plate portion 31, a range in which the hydrogen detector 20 is fixed is at least a flat plate shape.

In the flat plate portion 31, a collar portion 32 extends so as to surround the hydrogen detector 20. The collar portion 32 inclines downward as it moves away from the hydrogen detector 20. The collar portion 32 may be referred to as an eaves portion or the like. According to the first embodiment, the collar portion 32 extends from each of the four edges of the flat plate portion 31 and is connected as a whole, and surrounds the flat plate portion 31 and the hydrogen detector 20. In addition, the shape in which the end portion (the distal end 32a) of the collar portion 32 opposite to the end portion connected to the flat plate portion 31 is connected one round is a rectangular shape similar to the flat plate portion 31, which is a square or a rectangular shape close to a square.

As can be seen from FIG. 2, the hydrogen detector 20 and the cover 30 are arranged substantially concentrically about the hydrogen detector 20. Further, as can be seen from FIG. 3, the distal end 32a of the collar portion 32 is located farther from the ceiling surface 11 than the hydrogen detector 20. That is, the distal end 32a is located below the sensor unit 21 of the hydrogen detector 20.

Further, a plurality of through holes 34 are provided in at least one of the flat plate portion 31 and the collar portion 32. According to the first embodiment, each through hole 34 is formed in a slit shape along each of the four sides of the flat plate portion 31, and the plurality of through holes 34 are arranged so as to surround the hydrogen detector 20. The through hole 34 may be simply referred to as a slit. In FIG. 2, in the portion where the flat plate portion 31 and the collar portion 32 are connected, the through hole 34 is formed on the collar portion 32 side, but the through hole 34 may be formed on the flat plate portion 31 side, or may be formed over the flat plate portion 31 and the collar portion 32. Of course, the number of the through holes 34 is not limited to four as illustrated.

Further, a wall portion 35 protruding downward is provided on the lower surface 31a of the flat plate portion 31. The wall portion 35 extends to surround the hydrogen detector 20. In FIG. 2, the wall portion 35 surrounds the hydrogen detector 20 without interruption, but the continuity of the wall portion 35 may be partially interrupted around the hydrogen detector 20. According to FIG. 3, the wall portion 35 does not protrude downward from the distal end 32a of the collar portion 32. The wall portion 35 is provided between the hydrogen detector 20 and the through hole 34. However, the wall portion 35 may be located outside the hydrogen detector 20 with respect to at least a part of the through holes 34. Furthermore, the wall portion 35 may be provided on the lower surface of the collar portion 32.

The cover 30 according to the second embodiment will be described with reference to FIG. 4. FIG. 4 shows the cover 30 from a lower perspective, similar to FIG. 2. Only differences from the first embodiment will be described with respect to the second embodiment. A cross-sectional view of the second embodiment may be referred to FIG. 3 as in the first embodiment. As shown in FIG. 4, according to the second embodiment, the flat plate portion 31 and the collar portion 32 extending from the edge of the flat plate portion 31 have a circular shape. That is, the shape of the cover 30 when viewed from below may be circular.

In the second embodiment, each of the plurality of through holes 34 is also a slit curved along the curve of the edge of the flat plate portion 31, the circular formed by connecting the plurality of through holes 34, like the flat plate portion 31 and the collar portion 32, the hydrogen detector 20 It is concentric around. Further, according to FIG. 4, the wall portion 35 surrounding the hydrogen detector 20 is also concentric around the hydrogen detector 20.

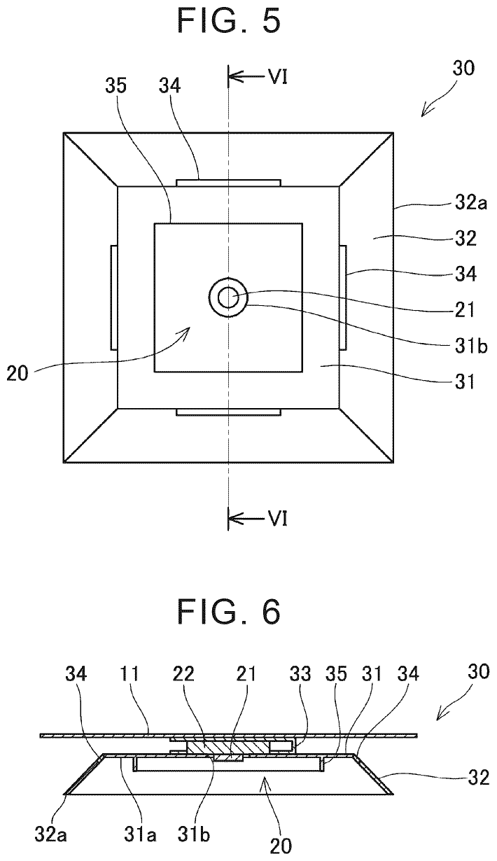

The cover 30 according to the third embodiment will be described with reference to FIGS. 5 and 6. FIG. 5 shows the cover 30 from a lower perspective, similar to FIGS. 2 and 4. FIG. 6 shows a cross-sectional view through VI-VI of FIG. 5. Also in the third embodiment, only the difference from the first embodiment will be described. According to the third embodiment, a through-hole 31b is formed in the center of the flat plate portion 31 so as not to allow the main body unit 22 of the hydrogen detector 20 to pass therethrough. The main body unit 22 is fixed on the flat plate portion 31, that is, in a space between the flat plate portion 31 and the ceiling surface 11.

The through-hole 31b is formed so as to allow the sensor unit 21 of the hydrogen detector 20 to pass therethrough, and the sensor unit 21 protrudes downward from the lower surface 31a via the through-hole 31b and faces downward. The support member 33 is shaped so as not to interfere with the main body unit 22 accommodated in the space between the flat plate portion 31 and the ceiling surface 11. Alternatively, the support member 33 and the main body unit 22 may be partially in contact with or connected to each other.

In this third embodiment, the position of the hydrogen detector 20 is closer to the ceiling surface 11 than in the first embodiment or the second embodiment. It can be said that the hydrogen detector 20 is advantageous to detect hydrogen by the amount close to the ceiling surface 11. Needless to say, in the third embodiment, the shape of the cover 30 may be a circular shape as shown in the second embodiment.

As described above, according to the present embodiment, the cover 30 for the gas detector (for example, the hydrogen detector 20) attached to the ceiling surface 11 includes the flat plate portion 31 disposed along the ceiling surface 11 and to which the gas detector is fixed, and the collar portion 32 extending so as to surround the gas detector in the flat plate portion 31 and inclining downward as the distance from the gas detector increases. According to the above configuration, when the leakage of the hydrogen gas 15 occurs in the space 10 as shown in FIG. 1, the hydrogen gas 15 drifting around the ceiling surface 11 can be held in the vicinity of the hydrogen detector 20 by the action of the collar portion 32 of the cover 30. As a result, the hydrogen concentration in the vicinity of the hydrogen detector 20 is stabilized, and the reliability of detection by one hydrogen detector 20 with respect to the leaked hydrogen gas 15 can be improved.

Further, according to the present embodiment, a plurality of through holes 34 may be provided in at least one of the flat plate portion 31 and the collar portion 32, and the plurality of through holes 34 may be arranged so as to surround the gas detector. According to the above configuration, even if the installation position of the hydrogen detector 20 is a position deviated from directly above the leakage position (for example, the joint 14) of the hydrogen gas 15, the hydrogen detector is ejected from the leakage position and then moves along the ceiling surface 11. The diffusing hydrogen gas 15 can enter the inside of the cover 30 from various directions through the respective through holes 34. As a result, the hydrogen concentration inside the cover 30, that is, in the vicinity of the hydrogen detector 20 is stabilized, and the reliability of the detection by the hydrogen detector 20 can be further increased.

Further, according to the present embodiment, the lower surface of the flat plate portion 31 may be provided with a wall portion 35 that protrudes downward and extends so as to surround the gas detector.

According to the above configuration, the effect of stabilizing the hydrogen concentration in the vicinity of the hydrogen detector 20 by holding the hydrogen gas 15 in the vicinity of the hydrogen detector 20 by the action of the wall portion 35 surrounding the periphery of the hydrogen detector 20 can be further enhanced.

Incidentally, the inclination angle θ (see FIG. 3) of the collar portion 32 with respect to the flat plate portion 31 may be set to, for example, 30 degrees to 60 degrees. If the inclination angle θ is too small, the effect of causing the hydrogen gas 15 to stay in the vicinity of the hydrogen detector 20 by the cover 30 is small. On the other hand, if the inclination angle θ is too large, the hydrogen gas 15 leaked at a position not directly under the hydrogen detector 20 cannot be efficiently guided to the inside of the cover 30. Therefore, a value range of 30 degrees to 60 degrees can be said to be an example of a value range suitable as the inclination angle θ.

Further, according to the present embodiment, the distal end 32a of the collar portion 32 may be located farther from the ceiling surface 11 than the gas detector. According to the above configuration, the distal end 32a of the collar portion 32 is located below the hydrogen detector 20. Therefore, it is possible to suppress the hydrogen gas 15 staying in the vicinity of the hydrogen detector 20 from flowing out of the cover 30 by the collar portion 32.

It is assumed that the hydrogen detector 20 is capable of detecting hydrogen having a predetermined concentration, for example, a concentration of 4% or more. When the hydrogen gas 15 leaked into the space 10 moves along the ceiling surface 11 after being raised, the hydrogen gas is diffused while meandering up, down, left, and right. Further, the hydrogen concentration decreases as the distance from the ceiling surface 11 decreases.

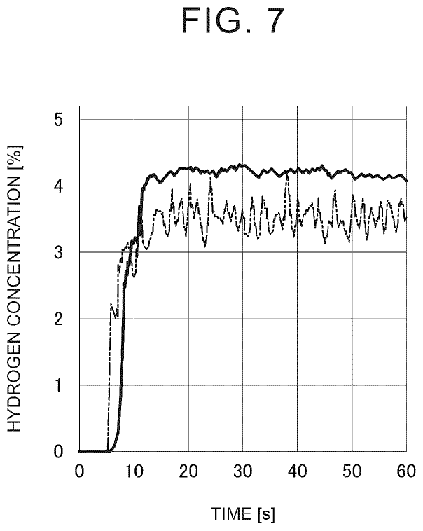

FIG. 7 graphically illustrates the change in hydrogen concentration over time in the vicinity of the hydrogen detector 20 at a predetermined position that is not directly above the leak position of the hydrogen gas 15, but a predetermined number of centimeters below the ceiling surface 11. In FIG. 7, the solid line graph shows the hydrogen concentration in the vicinity of the hydrogen detector 20 having the cover 30 of the first embodiment located at the predetermined position. On the other hand, in FIG. 7, a graph indicated by a two-dot chain line indicates the hydrogen concentration in the vicinity of the hydrogen detector 20 that is located at the predetermined position and does not include the cover 30. Note that the hydrogen concentration shown in FIG. 7 can be interpreted as a concentration obtained by using a predetermined detector having a higher detection capability than the hydrogen detector 20, which is different from the hydrogen detector 20, for experiment.

As can be seen from FIG. 7, according to the configuration without the cover 30, the variation in the hydrogen concentration is large and is less than 4% at many timings. On the other hand, according to the configuration having the cover 30, the hydrogen concentration is substantially stable at 4% or more. Therefore, in the conventional configuration without the cover 30, even if the leakage of the hydrogen gas 15 occurs, the hydrogen concentration is not stabilized in the vicinity of the hydrogen detector 20 that is not located directly above the leakage position, and the hydrogen detector 20 may not be able to detect hydrogen. On the other hand, according to the present embodiment, the hydrogen concentration in the vicinity of the hydrogen detector 20 is easily stabilized at a predetermined concentration or more by the action of the cover 30. Therefore, a situation in which the hydrogen detector 20 cannot detect hydrogen even though the hydrogen gas 15 leaks at any position in the space 10 can be eliminated as much as possible. In this way, in the present embodiment, since the hydrogen detector 20 is highly reliable in detecting the leakage of hydrogen, it is possible to carry out a wide range of hydrogen detection in one hydrogen detector 20, it is possible to contribute to cost reduction by reducing the number of necessary hydrogen detectors 20.

The shape of the cover 30 will be described. According to the drawings, the collar portion 32 extends from the edge of the flat plate portion 31 toward the side away from the hydrogen detector 20 and downward. However, the collar portion 32 may extend from a position closer to the hydrogen detector 20 than the edge of the flat plate portion 31 in the flat plate portion 31.

Further, a position where the collar portion 32 is connected to the flat plate portion 31 and a peripheral edge of the hydrogen detector 20 may be close to each other so that there is almost no distance. That is, the flat plate portion 31 may have a size equivalent to that of the hydrogen detector 20 as the minimum size required for fixing the hydrogen detector 20, and the collar portion 32 may be connected to the peripheral edge of the flat plate portion 31 having such a size.

The shape of the cover 30 when viewed from below is not limited to the shape shown in the drawing, and various shapes such as an elliptical shape and a polygon other than a quadrangle can be adopted.

While specific examples of the technology disclosed herein have been described in detail above, these are merely illustrative and do not limit the scope of the claims. Various modifications and variations of the specific examples described above are included in the technology described in the claims. In addition, the technical elements described in the present specification or the drawings exhibit technical usefulness alone or in various combinations, and are not limited to the combinations described in the claims at the time of filing. Further, the technology illustrated in the present specification or the drawings achieves a plurality of objects at the same time, and has technical usefulness by achieving one of the objects.

What is claimed is:

1. A cover for a gas detector attached to a ceiling surface, the cover comprising:
a flat plate portion that is disposed along the ceiling surface and to which the gas detector is fixed; and
a collar portion that extends to surround the gas detector on the flat plate portion and is inclined downward away from the gas detector.

2. The cover according to claim 1, wherein:
at least either of the flat plate portion and the collar portion has a plurality of through holes; and
the through holes are arranged to surround the gas detector.

3. The cover according to claim 1, wherein a lower surface of the flat plate portion includes a wall portion that protrudes downward and extends to surround the gas detector.

4. The cover according to claim 1, wherein an inclination angle of the collar portion with respect to the flat plate portion is 30 degrees to 60 degrees.

5. The cover according to claim 1, wherein a tip end of the collar portion is positioned farther from the ceiling surface than the gas detector.
